(19) United States
(12) Patent Application Publication
(10) Pub. No.: US 2007/0161102 A1
(43) Pub. Date: Jul. 12, 2007

(54) Microchannel biosensor strip capable of simultaneously detecting different human physiological information

(75) Inventors: Paul Yang, Taipei (TW); Jen-Chien Chien, Yilan County (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, CHUNG HO CITY (TW)

(21) Appl. No.: 11/327,330

(22) Filed: Jan. 9, 2006

(51) Int. Cl. C12M 1/34

(52) U.S. Cl. 435/287.1; 205/777.5

(57) ABSTRACT

This invention discloses a microchannel biosensor strip capable of simultaneously detecting different human physiological information, and particularly a biosensor strip structure has a plurality of microchannels, such that various physiological information can be detected by human body fluid (such as blood), and the test strip has a plurality of microchannels, such that the human body fluid can be guided by a capillary diffusion phenomenon to different biological enzyme reacting areas connected to the microchannel for the reaction, and the content of biological matters of different testing items including glucose, total cholesterol, high density lipoprotein, and uric acid can be detected successively or simultaneously.

### BACKGROUND OF THE INVENTION

1. Field of the Invention

The present invention relates to a microchannel biosensor strip capable of simultaneously detecting different human physiological information, and more particularly to a biosensor strip having a plurality of microchannels for detecting various different human physiological information by a human body fluid (such as blood), and the biosensor strip includes a plurality of microchannels that guide the blood to different enzyme reacting areas connected to the microchannels by a capillary diffusion phenomenon, and the blood is reacted with different reacting enzymes disposed in their respective enzyme reacting areas, so as to detect the contents of several different biochemical items simultaneously or one by one.

2. Description of the Related Art

In recent years, some chronic illnesses including diabetes, high blood pressure, hyperglycemia, and gout, etc threaten our health as the economic conditions and level of living standards are improved rapidly. In addition to the diet control and appropriate exercises, people also need to go to a hospital for medical examinations periodically, so that they can know about their current health conditions.

Since the concept of household nursing becomes increasingly popular, and there are different kinds of self testing products for a fast, cheap, non-professional operation, and these sensor products including a blood sugar meter, an electronic ear thermometer, an electronic sphygmomanometer, and a cholesterol measuring device, etc waste no time or money to go to a hospital or require no assistance of medial people to carry out the measurements. Users just need to have their own medical measuring instruments to know about their health conditions anytime and anywhere, so as to prevent any possible delay of medical treatments. Since diabetes, high blood pressure, hyperglycemia, and gout are closely related to each other and may begin at the same time, therefore users usually need to take several different tests before fully knowing about the patient's conditions.

Referring to FIG. 1, most examinations of the general chronic illnesses are conducted by analyzing the patient's blood, and users need to take a blood sample from the patient's finger or earlobe, and the blood is placed at an opening 101 of an electrode test strip 100 of a biosensor. The opening 101 has a reacting enzyme area 102 for the reaction, and each different reacting enzyme is provided for testing one of the biological information only. The electrode test strip 100 of a prior art biosensor is made of a sheet insulating substrate which is generally an insulating and heat resisting substrate to facilitate the heating and solidification of the biological reacting film and electric conductive layer, such as a PC board, a PVC board, a PP board, a glass fiber board, a bakelite board, a ceramic board, or a glass board. The electric conductive layer includes a pair of separate electrode signal circuits 103, one being a working electrode signal circuit and the other being a reference electrode signal circuit. One end of the electrode signal circuit is coupled to the enzyme reacting area 102, and the other end of the electrode signal circuit is coupled to an electrochemical biosensor (not shown in the figure), such that the biological information in the blood can be detected by an electrochemical reaction. However, the test requires a separate blood sample for each test item each time, and taking blood sample each time means a pain to the patient for one time. Particularly, patients having a chronic illness have to take the test and bear with the pain repeatedly. Since the electrode test strip 100 for testing human biological information such as the level of glucose has only one testing object disposing opening 101 and only one biological reacting enzyme. If a user wants to know about other human biological information, such as the content of the total cholesterol, high density lipoprotein, low density lipoprotein, or uric acid, it is necessary for users to take the blood samples again and use another electrode test strip and biosensor for the test.

### SUMMARY OF THE INVENTION

It is a primary objective of the present invention to provide a microchannel biosensor strip capable of simultaneously detecting different human physiological information and particularly a biosensor strip structure has a plurality of microchannels, such that various physiological information can be detected by human body fluid (such as blood), and the strip has a plurality of microchannels, such that the human body fluid can be guided by a capillary diffusion phenomenon to different biological enzyme reacting areas connected to the microchannel for the reaction, and the content of biological matters of different testing items including glucose, total cholesterol, high density lipoprotein, and uric acid can be detected successively or simultaneously.

### BRIEF DESCRIPTION OF THE DRAWINGS

FIG. 1 is a schematic view of an electrode test strip of a prior art biosensor;

FIG. 2 is a schematic view of a first preferred embodiment of the present invention;

FIG. 3 is an exploded view of a first preferred embodiment of the present invention;

FIG. 4 is a schematic view of a second preferred embodiment of the present invention;

FIG. 5 is a cross-sectional view of a second preferred embodiment of the present invention; and

FIG. 6 is a schematic view of using the present invention.

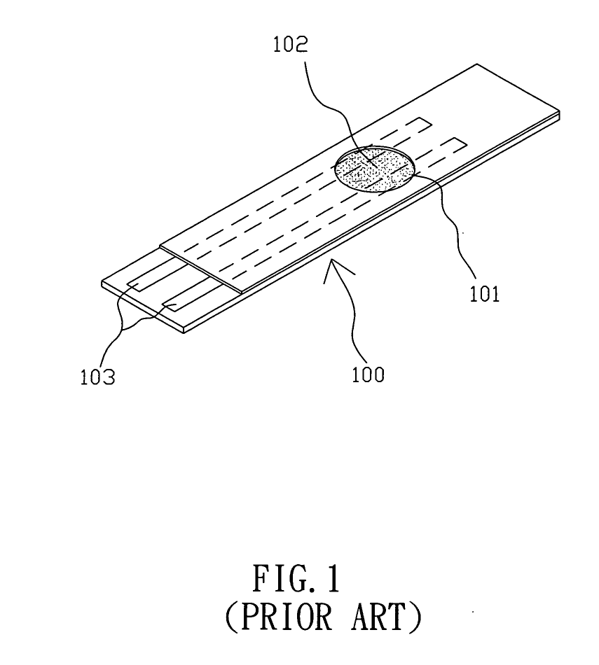

### DETAILED DESCRIPTION OF THE PREFERRED EMBODIMENTS

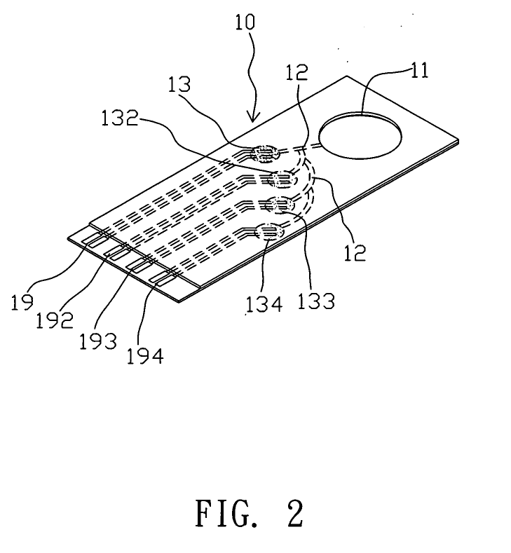

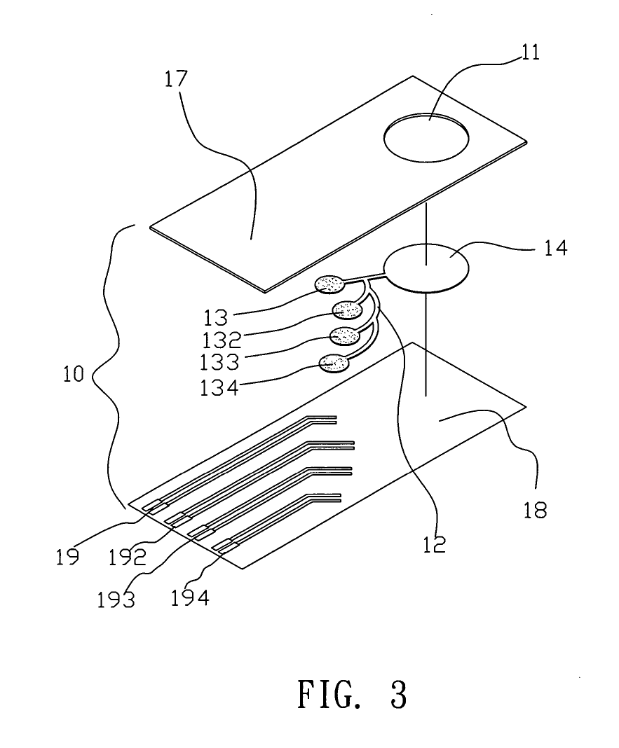

Referring to FIGS. 2 and 3 for the schematic view and the exploded view of a first preferred embodiment of the present invention, four test items of human biological information can be detected simultaneously, but the biosensor strip can build the detecting areas for different testing items according to practical needs. The structure of a microchannel biosensor strip capable of simultaneously detecting different human physiological information is an electrode test strip 10 comprising a cover plate 17, and the cover plate 17 at its side includes a testing object disposing opening 11, and the testing object disposing opening 11 is corresponsive to a testing object disposing area 14, and the testing object disposing area 14 is coupled to a microchannel 12, and the microchannel 12 at its end has a first enzyme reacting area 13, and the microchannels 12 are coupled to the second enzyme reacting area 132, third enzyme reacting area 133, and fourth enzyme reacting area 134 in sequence, wherein the first enzyme reacting area 13, second enzyme reacting area 132, third enzyme reacting area 133, and fourth enzyme reacting area 134 could be separated as a glucose enzyme reacting area, a total cholesterol enzyme reacting area, a high density lipoprotein enzyme reacting area, or a uric acid reacting area according to different reacting enzymes. The substrate 18 is an insulating slab having a first pair of working and reference electrodes 19, a second pair of working and reference electrodes 192, a third pair of working and reference electrodes 193, and a fourth pair of working and reference electrodes 194. The first enzyme reacting area 13 is coupled to a first pair of working and reference electrodes 19, and the second enzyme reacting area 132 is coupled to the second pair of working and reference electrodes 192, and the third enzyme reacting area 133 is coupled to the third pair of working and reference electrodes 193, and the fourth enzyme reacting area 134 is coupled to said fourth pair of working and reference electrodes 194, such that the electrochemical reactions in the first enzyme reacting area 13, second enzyme reacting area 132, third enzyme reacting area 133, and fourth enzyme reacting area 134 can be carried out by the first pair of working and reference electrodes 19, second pair of working and reference electrodes 192, third pair of working and reference electrodes 193, and fourth pair of working and reference electrodes 194.

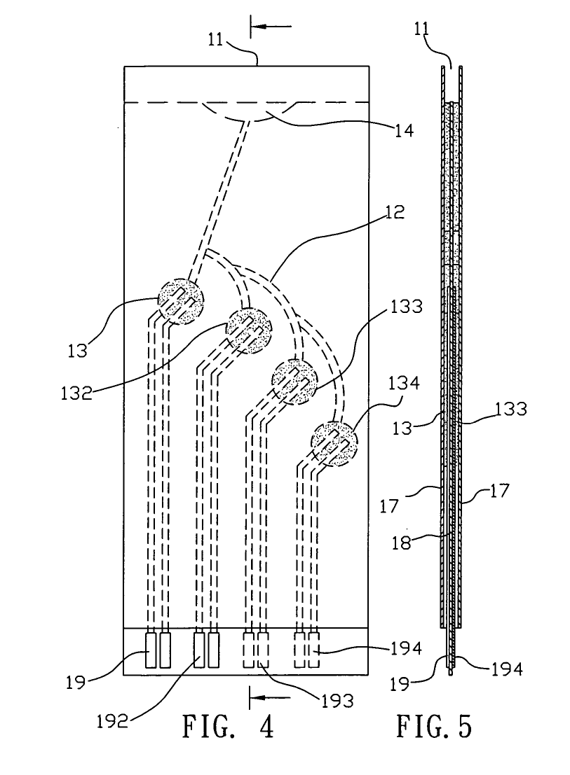

Referring to FIGS. 4 and 5 for the schematic view and the cross-sectional view of another preferred embodiment of the present invention, the preferred embodiment adopts a three-dimensional electrochemical test strip, wherein the first enzyme reacting area 13 with the first pair of working and reference electrodes 19 and the second enzyme reacting area 132 with the second pair of working and reference electrodes 192 are disposed on one side of the substrate 18, and the third enzyme reacting area 133 with the third pair of working and reference electrodes 193 and the fourth enzyme reacting area 134 with the fourth pair of working and reference electrodes 194 are disposed on another side of the substrate 18, and thus the width of the electrode test strip can be reduced.

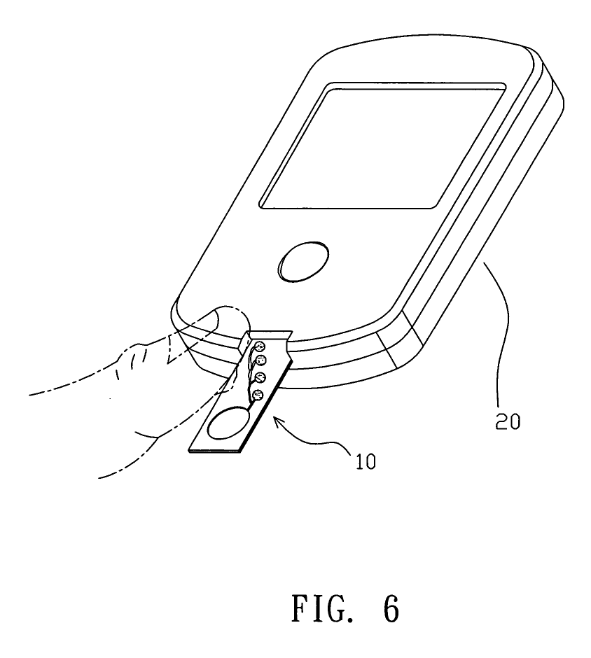

Referring to FIG. 6 for the schematic view of using a human biological signal sensor 20 of the microchannel biosensor strip 10 to read the content of biological matters according to the present invention, a user inserts the electrode test strip 10 into the human biological signal sensor 20 and then puts in the testing object. The computation and processing of the human biological signal sensor 20 will show related human biological information such as the level of glucose, total cholesterol, high density lipoprotein, or uric acid, after the testing object is tested.

In summation of the description above, the present invention adopts a plurality of microchannels and uses the capillary diffusion phenomenon to send the blood in the testing object from the opening to different enzyme reacting areas to simultaneously measure several test values of the human biological information and provide a more economic and efficient test.

While the invention has been described by means of specific embodiments, numerous modifications and variations could be made thereto by those skilled in the art without departing from the scope and spirit of the invention set forth in the claims.

What is claimed is:

1. A microchannel biosensor strip capable of simultaneously detecting different human physiological information for detecting the content of biological matters of
a testing object, comprising:
an insulating substrate;
a cover plate, corresponding and coupled to said cover plate and having a testing object disposing opening disposed at an end of said cover plate;
a testing object disposing area, corresponding to said testing object disposing opening;
a plurality of microchannels, coupled with each other, and one end of said each microchannel being coupled to said testing object disposing area and another end coupled to said each enzyme reacting area; and
at least one working and reference circuit, attached onto said substrate and having an end coupled to said enzyme reacting area, and another end extended to an end of said strip, and capable of detecting at least one human biological information.

2. The microchannel biosensor strip capable of simultaneously detecting different human physiological information of claim 1, wherein said microchannels are coupled to each enzyme reacting area progressively.

3. The microchannel biosensor strip capable of simultaneously detecting different human physiological information of claim 1, wherein said microchannels maintain equidistant with said each enzyme reacting area.

4. The microchannel biosensor strip capable of simultaneously detecting different human physiological information of claims 2 or 3, wherein said microchannels coupled with said working and reference circuit are disposed on both sides of said substrate.
